(19) United States
(12) Patent Application Publication
(10) Pub. No.: US 2011/0217350 A1
(43) Pub. Date: Sep. 8, 2011

(54) SUBSTRATE HAVING A SURFACE FOR INHIBITING ADHESION OF A TARGET CELL THEREON AND A METHOD OF PREPARING THE SAME

(75) Inventors: Isabel Rodríguez Fernández, Singapore (SG); Li Buay Koh, Singapore (SG)

(73) Assignee: AGENCY FOR SCIENCE, TECHNOLOGY AND RESEARCH, Singapore (SG)

(21) Appl. No.: 13/119,207

(22) PCT Filed: Sep. 24, 2009
(86) PCT No.: PCT/SG09/00354

§ 371 Date: May 24, 2011

Related U.S. Application Data
(60) Provisional application No. 61/099,754, filed on Sep. 24, 2008.

(51) Int. Cl. A61F 2/00; A61K 9/00; B28B 11/08

(52) U.S. Cl. 424/422; 424/400; 264/293

(57) ABSTRACT

A substrate having a surface for inhibiting adhesion of a target cell thereon, the substrate comprising an array of generally longitudinal projections having a longitudinal axis that extends from the surface of said substrate and having an aspect ratio of at least 2.5, wherein adjacent projections of said array are configured on the substrate such that the projections at least partially inhibit adhesion of a target cell thereon.

### TECHNICAL FIELD

The present invention generally relates to a substrate having a surface for inhibiting adhesion of a target cell thereon. The present invention also relates to a method of preparing the surface of a substrate to inhibit adhesion of a target cell thereon.

### BACKGROUND

When a medical device is implanted into a human body, there is risk of rejection by the human body, which may take place in the form of fibrosis, thrombosis and inflammation and may be complicated with bacterial infection. In particular, the implantation of blood-contacting medical devices can lead to thrombosis at the blood-material interface with the associated risk of thromboembolic events.

It is widely recognized that when a foreign material is placed in contact with the human body, it rapidly adsorbs a layer of proteins and a series of events mediated by these proteins onto specific cells leads to processes such as thrombosis, inflammation or rejection. The extent of these reactions depends on the biocompatibility of the biomaterial.

In the case of blood contacting devices, adsorption of proteins, such as fibrinogen, to the material may lead to adhesion and activation of the plasma platelets and the consequent activation of the coagulation cascade, resulting in the production of thrombin, an important enzyme that leads to formation of fibrin polymer strands, as well as a potent activator of platelets. Adherent platelets become activated, secreting agonists that activate additional platelets, and providing a substrate for promulgation of the coagulation cascade. These intertwined events lead to the formation of a stable thrombus on the material surface, consisting of activated platelets adherent to each other and to the material, strengthened by fibrin polymer strands.

Materials that reduce the adhesion and activation of platelets could therefore facilitate the development of medical devices with improved hemocompatibility. A variety of biocompatible polymeric materials are used in blood contacting medical devices. To render their use in such applications, these materials may undergo surface passivation processes to increase their bio or hemo-compatibility. Examples of surface passivation processes include coating with a carbon based inorganic coating, bioactive coating using fragments of biomacromolecules, formation of a covalently linked coating layer by employing brushes of long-chained hydrophilic molecules like poly-ethylene oxide, chemical composition modification of polymer surfaces, immobilization of biological molecules with controllable positioning and size, ion implantation and graft polymerization. However, one of the limitations in using coatings is its delamination under shear stress in blood flow, thereby causing complications downstream. In addition, the cost and complexity of these coatings could further complicate the regulatory approval process. Nonetheless, none of the artificial surfaces developed to-date is completely inert towards blood. To avoid thrombosis or thromboembolism complications, patients with medical implants have to undergo anticoagulant therapy, which in turn leads to undesirable side effects.

An alternative to using chemically-based modification of surfaces is to create topographic structures on the surface of the blood contacting medical devices. Such structures can be created by using colloidal-derived topographies and polymer demixing of an immiscible polymer blend. However, the structures formed using these processes are typically random structures having inconsistent heights and widths between batch-to-batch devices, leading to problems of reproducibility and uncertainty associated with the level of platelet adhesion. Another way of modifying the topography of a surface of a medical surface is to form dual-scale structures with nano and micron dimensions. However, the level of platelet adhesion on the structured surface, while lower than that on a pristine surface, is not within the desired range. A further known substrate having nanoscale structures on the surface of a substrate has been taught in the art, however the problem with these nanoscale structures is that they do not significantly decrease the level of platelet adhesion on the structured surface because there is no control over the dimension and displacement of the structures on the substrate surface. Further, this known substrate was produced using a process which was not able to result in the miniaturization of the topographic structures in order to arrive at a low level of platelet adhesion. While efforts have been made to decrease the dimensions and parameters of the topographic structures to the nano-meter scale in order to decrease the level of platelet adhesion on the medical device, these topographic structures are not able to achieve a desired low level of platelet adhesion.

Therefore, there is a need for an improved substrate having topographic structures of desired parameters in order to achieve a desired low level of adhesion of a target cell thereon.

There is also a need to provide a method that can produce a substrate having topographic structures of the desired parameters that overcomes, or at least ameliorates, one or more of the disadvantages described above.

### SUMMARY

According to a first aspect, there is provided a substrate having a surface for inhibiting adhesion of a target cell thereon, the substrate comprising an array of generally longitudinal projections having a longitudinal axis that extends from the surface of said substrate and having an aspect ratio of at least 2.5, wherein adjacent projections of said array are configured on the substrate such that the projections at least partially inhibit adhesion of a target cell thereon.

Advantageously, when the aspect ratio of the projections is above 2.5, the projections substantially inhibit adhesion of target cells on the substrate. For example, platelets, adhering to the substrate surface are substantially reduced as compared to substrates that have such projections that do not fall within the defined aspect ratio. In one embodiment, the aspect ratio of said projections is in the range of 3 to 20. The inventors have also discovered that this selected range of aspect ratio is the optimal range for inhibiting cell adhesion. Without being bound by theory, it is believed that beyond the upper limit of the range, clustering of the projections may occur such that an increased surface area is available for cellular contact, which in turn increases cell adhesion to the substrate.

When the aspect ratio is below 2.5, the target cells are not adequately inhibited from adhering thereon because the projections are not as effective to prevent contact of the target cells with the underlying substrate. Furthermore, the projections become relatively inflexible when submerged in a a fluid environment which increases the likelihood of establishing stable contact between the target cells and projections or underlying substrate.

In one embodiment, the adjacent projections of said array are disposed from each other at a distance less than the diameter of the target cell. Advantageously, as the distance of adjacent projections is less than the diameter of the cells, the likelihood of the cells being able to adhere to the surface within inter-projection spacing is largely reduced. This is due to the size exclusion effect of the inter-projection spacing, that is, the cell is too large to be able to get into the spacings between adjacent projections.

In one embodiment, the adjacent projections of said array are disposed from each other at a distance of 200 nm or less and with an aspect ratio 2.5 and above. Advantageously, this largely reduced inter-projection spacing and aspect ratio not only excludes the entire cell from entering the spaces between adjacent cells but also reduces the possibilities of pseudopods or extensions from the cell to make stable contacts to the projections surface and the surface within the inter-projection spaces. This aids in the reduction of adhesion of cells, especially motile cells, onto the surfaces of the substrate. It has also been surprisingly discovered by the inventors that the substrate is extremely effective to inhibit cell adhesion, specifically platelet adhesion, when the projections are disposed from each other at a distance of 200 nm or less as compared to other inter-projection distances.

In one embodiment, at least one of the substrate and projections is comprised of a biocompatible polymer. Advantageously, the substrate can be used in applications that require contact with the patient's body without eliciting an adverse immune response from the patient. For example, the substrate may be an implant that is placed for long periods of time in the patient's body or a catheter or a tube that would be in constant contact with the patient's bodily fluids in vivo or ex vivo.

In one embodiment, the biocompatible polymer is a thermoplastic. Advantageously, when the biocompatible is a thermoplastic, techniques such as casting or nanoimprinting can be easily utilized to form the projections on the substrate. More advantageously, thermoplastics can be easily reshaped into projections with high aspect ratio nanostructures and hence the projections on the substrates can be reshaped and customized as and when the need arises. In one embodiment, the thermoplastic is selected from the group consisting of poly(D,L-lactic acid) (PDLLA), polyglycolic acid (PGA), poly(3-hydroxybutyrate) (PHB), polycarbonate (PC), poly(lactide-co-glycolic acid) (PLGA) Polycaprolactone (PCL), Poly(ethylene terephthalate) (PET), Polypropylene (PP), Polyurethanes (PU), Poly(vinyl chloride) (PVC) Polysulfones (PS) Polyamides (PA) Polyacetal (POM), Polyacrylonitrile (PAN), Poly(tetrafluoroethylene) (PTFE), thermosetting polymers like polydimethylsiloxane (PDMS), epoxies and combinations thereof.

In one embodiment, the substrate as claimed in any one of the preceding claims, wherein said target cell is selected from the group consisting of thrombocytes, macrophages, dendritic cells, lymphocytes, granulocytes and bacteria. Advantageously, when the substrates are used in applications that require contact with the blood, the cells present in the blood such as thrombocytes, macrophages, dendritic cells, lymphocytes and granulocytes which elicit blood clotting and immune responses in the patient's body can be inhibited from adhering to the substrate. More advantageously, this reduces the likelihood of complications such as foreign body rejection, thrombosis, embolism or infection in the patient.

In one embodiment, the projections are flexible in that at least part of each projection is capable of deflecting from its longitudinal axis when subjected to force. Advantageously, when the substrate is placed in a fluid medium that has fluid motion, the projections are able to move about their points of attachment to the surface by deflection off their longitudinal axes. More advantageously, this sweeping movement of the projections when placed in the path of a moving fluid aids in inhibiting cell adhesion on the surface of the substrate. While not being bound by theory, it is believed that such sweeping movements of the projections prevent the cells from adhering stably to the surfaces of the substrate. The sweeping movements also advantageously, encourage detachments of any weakly attached cells on the substrate. Thus, when the substrates are used in applications that require contact with the blood flow of the patient, such as catheters and intravascular tubes, the flow of blood would initiate the sweeping movements of the projections to thereby aid in inhibiting platelet adhesion on the substrate. More advantageously, clot formation on the substrate is inhibited and hence preventing the impairment of the substrate's functionality as well as possible medical complications that may occur due to the presence of blood clots. In one embodiment, the longitudinal axes of the projections are disposed at a substantially normal angle relative to a planar surface of said substrate.

In one embodiment, the projections have heights in the range of from 300 nm to 1000 nm. In another embodiment, the projections have widths in the range of from 50 nm to 300 nm. It has been surprisingly discovered by the inventors that this specific range of height and width of the projections ensures optimal cell inhibition. More advantageously, this specific range of height and width allows the projections to have sufficient mechanical strength to withstand oncoming fluid flow without breaking but at the same time also be able to maintain the required amount of flexibility to inhibit cell adhesion thereon.

In a second aspect, there is provided an implantation device for implantation into a patient's body, wherein the device comprises a surface that has an array of longitudinal projections extending from a surface of said device and wherein said projections have an aspect ratio of at least 2.5, wherein adjacent projections of said array are configured on the surface of the device such that the projections at least partially inhibit adhesion of a target cell thereon.

In a third aspect, there is provided a method of inhibiting adhesion of a target cell on a device in contact with body fluids, the method comprising the steps of:

providing a device comprising a substrate having an array of longitudinal projections extending from the surface of said substrate and having an aspect ratio of at least 2.5, wherein adjacent projections of said array are configured on the substrate such that the projections at least partially inhibit adhesion of a target cell thereon; and

contacting the body fluids with the substrate.

In a fourth aspect, there is provided a method of preparing a surface of a substrate, the method comprising the step of forming an array of longitudinal projections that extend from the surface of said substrate and have an aspect ratio of at least 2.5, wherein adjacent projections of said array are configured on the substrate to at least partially inhibit adhesion of a target cell thereon. In one embodiment, the step of forming an array of longitudinal projections comprises at least one of nanoimprinting, capillary force imprinting and casting. Advantageously, these methods are capable of producing projections on the substrate of dimensions up to the nanometer range and thus can be utilized to produce the preferred embodiment of the substrate disclosed herein. In one embodiment, the step of forming the array of longitudinal projections may optionally exclude the step of chemically treating the surface.

In one embodiment, the step of forming an array of longitudinal projections comprises the step of nanoimprinting the projections on the substrate.

In another embodiment, the step of forming an array of longitudinal projections comprises the steps of:

(a) providing a mold having longitudinal cavities extending therethrough;

(b) applying a thermoplastic material to the mold and allowing the thermoplastic material to enter said longitudinal cavities under capillary force to thereby produce said projections on the substrate. The temperature of the thermoplastic material during said applying step may be above the glass transition temperature of the thermoplastic material.

In another embodiment, the step of forming an array of longitudinal projections comprises the step of

(a) providing a mold having longitudinal cavities extending therethrough;

(b) applying a polymerizable solution to the mold and allowing said polymerizable solution to enter said longitudinal cavities;

(c) heating the polymerizable solution after said applying step (b), to thereby produce said projections on the substrate upon demolding.

In another embodiment, the step of forming an array of longitudinal projections comprises the step of

(a) providing a mold having longitudinal cavities extending therethrough;

(b) applying a polymer solution to the mold and allowing said polymerizable solution to enter said longitudinal cavities;

(c) evaporating the solvent in the polymer solution after said applying step (b), to thereby produce said projections on the substrate upon-demolding.

### DEFINITIONS

The following words and terms used herein shall have the meaning indicated:

The term “target cell” as used herein broadly refers to any cell that is intended to be inhibited from adhering to the surface of a substrate. The target cells may be eukaryotic cells or prokaryotic cell. Exemplary eukaryotic cells include but are not limited to, platelets, erythrocytes, leukocytes, macrophages, dendritic cells, fibroblasts, granulocytes and lymphocytes. Exemplary prokaryotic cells include but are not limited to bacteria.

The term “aspect ratio” as used herein is to be interpreted broadly to refer to the ratio of the greatest dimension, generally the length or height, to the width of a structure. Thus, when the term “aspect ratio” is used to describe the projections extending from a surface, it should be interpreted to mean the ratio of the height of the projections to the width of the projection or the diameter of the projection if the cross-sectional area of the projection is circular in shape.

The term “at least partially inhibit adhesion” or grammatical variants thereof, as used herein is meant to be interpreted broadly to include preventing adhesion as well as discouraging or reducing adhesion of the target cells on a substrate. Hence, when the projections disclosed herein are described as-being able to “at least partially inhibit adhesion” of target cells on a substrate, it should be interpreted that the projections are capable of being able to entirely prevent adhesion of target cells on the substrate or at least reduce the number of cells adhering to the surface when compared to a substrate that does not comprise said projections; that is a substrate that is absent of projections.

The term “low adhesion” in the context of this specification, refers to the adhesion of target cells in a sample that is comparable to, or lower than, the number of target cells in the same sample that adheres to a substrate having carbon nanotubes thereon.

The term “patient” as used herein is to be interpreted broadly to include a human or an animal patient.

The terms “inter-pillar spacing”, “inter-structural spacing”, “inter-projections spacing” used herein are to be interpreted to mean the distance, area, volume or void as may be appropriate, between adjacent pillars, structures and projections respectively. Likewise variants of such terms are to be construed in the same manner accordingly. For example, the term “interspacing” can be used interchangeably with term “inter-pillar spacing” or “inter-projection spacing” or “inter-structural spacing” as may be appropriate.

The term “biocompatible” as used herein refers to any material which does not cause severe toxicity, severe adverse biological reaction, severe adverse immunological reaction or lethality in an animal or human when administered at reasonable amounts.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DISCLOSURE OF EMBODIMENTS

Exemplary, non-limiting embodiments of a substrate having an array of longitudinal projections extending from the surface of said substrate to at least partially inhibit adhesion of a target cell on said substrate, will now be disclosed.

The substrate has a surface for inhibiting adhesion of a target cell thereon and the substrate comprises an array of longitudinal projections extending from the surface of said substrate and having an aspect ratio of at least 2.5, wherein adjacent projections of the array are configured on the substrate such that the projections at least partially inhibit adhesion of a target cell thereon.

The aspect ratios of the projections may be at least about 3, at least about 5, at least about 8, at least about 10, at least about 12 and at least about 14. In one embodiment, the aspect ratio of the projections is no more than about 20, no more than about 18 and no more than about 15. In one embodiment, the aspect ratio of said projections is in the range of 3 to 20. Preferably, the aspect ratio of the projections is from about 8 to about 15.

The projections may be nano-sized structures or micro-sized structures. Preferably, the projections are nano-sized structures. When the projections are nano-sized structures, the height of the nano-structures may be in the range of from about 340 nm to about 8000 nm, from about 300 nm to about 7000 nm, from about 300 nm to about 6000 nm, from about 300 nm to about 5000 nm, from about 300 nm to about 4000 nm, from about 300 nm to about 3000 nm, from about 300 nm to about 2000 nm, from about 300 nm to about 1000 nm, from about 400 nm to about 1000 nm, from about 500 nm to about 800 nm, from about 600 nm to about 800 nm or from 700 nm to about 800 nm. In one embodiment, the projections have heights in the range of from about 700 nm to about 900 nm.

The width of the nano-structures may be in the range of from about 50 nm to about 800 nm, from about 50 nm to about 200 nm, from about 50 nm to about 100 nm, from about 50 nm to about 80 nm, from about 100 nm to about 300 nm, from about 100 nm to about 250 nm, from about 100 nm to about 200 nm or from about 100 nm to about 150 nm. In one embodiment, the projections have widths in the range of from about 70 nm to about 130 nm.

In one embodiment, the adjacent projections of said array are disposed from each other at a distance less than the diameter of the target cell. This ensures that spacing between 2 adjacent projections is smaller than the diameter of the cell to prevent the cell from adhering within the inter-projection spacing. In another embodiment, the density of the projections in the array is at least 2 structures per cell area. In one embodiment, the spacing between adjacent projections is in the range of from about 100 nm to about 500 nm, from about 100 nm to about 400 nm, from about 100 nm to about 300 nm and from about 100 nm to about 200 nm. Preferably, the spacing between adjacent projections is less than about 200 nm, less than about 180-nm, less than about 160 nm, less than about 150 nm or less than about 140 nm.

In one embodiment, the projections are flexible. The projections may be flexible such that at least part of each of the projections is able to deflect from their longitudinal axis when placed in the path of a moving fluid. For example, when the projections are placed in a path of a fluid medium which is in motion, the projections may be able to move about their points of attachment from the base to deflect from its longitudinal axis, in a sweeping/swaying manner. When the fluid medium is no longer in motion, the projections may also be able to return back to their original positions and realign with their longitudinal axis.

The projection may be of any cross-section, including circular, oval, square, rectangular, polygonal etc. In one embodiment, the projections are pillars extending from the surface of the substrate. The pillar cross-section may be substantially uniform along most or substantially all of the height of the pillar, or may taper as the pillar extends away from the rest of the surface. The pillar side walls may be generally orthogonal to the surrounding surface. The pillar may also have a generally flat or a rounded cap.

In one embodiment, the projections are presented in an array, which may be in any chosen pattern, such as a square pattern, a rectangular pattern, a circular pattern, a rosette pattern, a random pattern etc. The array of projections may also comprise of projections having different dimensions, for example different widths and heights, as long as the aspect ratios, height and widths fall within the specified ranges defined above.

The substrate and/or the projections may be comprised of polymers such as synthetic polymers, polymeric biomaterials, biopolymers, and other polymers. In one embodiment, the substrate and/or the projections are comprised of a biocompatible polymer. The biocompatible polymer may, if desired, be formed of a resorbable material which can be resorbed into the living tissue within a certain period of time. For example, if the substrate is intended to prevent adhesion of tissue structures during a healing process, the substrate and projections can be comprised of resorbable material which is resorbed after fulfilling its function. In addition, the biocompatible polymer may be a thermoplastic. In one embodiment, the thermoplastic is selected from the group consisting of poly(D,L-lactic acid) (PDLLA), polyglycolic acid (PGA), poly(3-hydroxybutyrate) (PHB), polycarbonate (PC), poly(lactide-co-glycolic acid) (PLGA) Polycaprolactone (PCL), Poly(ethylene terephthalate) (PET), Polypropylene (PP), Polyurethanes (PU), Poly(vinyl chloride) (PVC) Polysulfones (PS) Polyamides (PA) Polyacetal (POM), Polyacrylonitrile (PAN), Poly(tetrafluoroethylene) (PTFE), thermosetting polymers like polydimethylsiloxane (PDMS), epoxies and combinations thereof.

The target cell disclosed herein may be selected from the group consisting of thrombocytes, macrophages, dendritic cells, lymphocytes, granulocytes, bacteria or any cells that are capable of adhering to the substrate without the disclosed projections.

The substrate may be any substrate that encounters a biological environment whether by implantation, extracorporeal processing of blood or other body fluids, or substrates used in food and beverage handling and work surfaces that encounter biological formed elements such as bacteria. For example the substrate may be an implant, a graft and a prosthesis introduced into the vascular system, gastrointestinal tract, lung alveolae, synovia, connective tissue and in the eye or gum and in any part of the body. In one embodiment, the substrate is a catheter which is in contact with the patient's blood. In another embodiment, the substrate is an implantation device such as a stent, heart pacer or artificial heart valve for implantation into a patient's body.

The array of projections extending from the surface of the substrates can be formed at least one of nanoimprinting, capillary force imprinting, template wetting and casting. In one embodiment, when thermoplastic nanoimprinting is used, the nanoimprinting is carried out at a temperature range above the glass transition temperature of the polymer and in the pressure range of about from about 10 Bar to 80 Bar. In one embodiment, when photo nanoimprinting is used, a photo(UV) curable liquid pre-polymer is applied to a substrate. A mold, typically made of transparent material, may then be pressed together and the pre-polymer may be cured in UV light to crosslink and solidify. After which, the mold may be separated to release the structures. In another embodiment when template wetting is used, the template wetting is carried out in the temperature range of well above the glass transition temperature of the polymer and in the pressure range of about from about 100 mBar to 5000 mBar. In another embodiment, when casting is used, the process of casting is carried out at room temperature and in vacuum.

In one embodiment, after the array of projections has been formed on the substrate, the projections and/or the surface of the substrate can be further chemically enhanced or functionalized to confer additional anti-adhesion properties to the substrate. The projections and/or the surface of the substrate may also be made to increase their hydrophobicity, which may enhance inhibition of adhesion a target cell thereon.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1a is a quantification graph showing the amount of fibrinogen adsorbed on the series of test substrates, control substrates and reference sample as determined from the calibrated curve by ELISA. FIG. 1b is a quantification graph showing the number of platelets adherent on the test substrates on FIG. 1a as determined from the calibrated curve by the LDH assay. FIG. 1c is a quantification graph showing the PAC-1 expression on platelet activation on the test substrates of FIG. 1a derived from flow cytometric analysis.

FIG. 2a is a scatter diagram which was tabulated from FIG. 1a and FIG. 1b showing a strong correlation on the amount of adsorbed fibrinogen by ELISA against the number of adherent platelets by the LDH assay for the series of topographic projections tested.

FIG. 2b is a scatter diagram which was tabulated from FIG. 1b and FIG. 1c showing a strong correlation the number of adherent platelets by the LDH assay against the PAC-1 expression of activated platelets as determined by flow cytometric analysis.

FIG. 3a is a forward versus side light scatter dot plot with mean intensities for stained platelet-bound fibrinogen obtained through gating displaying platelets only (positive control). FIG. 3b is a forward versus side light scatter dot plot with mean intensities for stained platelet-bound fibrinogen obtained through gating displaying higher platelet activation on the pristine PLGA. FIG. 3c is a forward versus side light scatter dot plot with showing a lower mean intensity for stained platelet-bound fibrinogen obtained through gating for P(1,2.5,8) sample.

FIG. 4a is an SEM image obtained at 500× magnification of a comparison between a pristine PLGA surface and a PLGA surface structured with pillars having an interspacing of about 150 nm, showing a higher level of platelets adhesion on the pristine surface as compared to the structured surface. FIG. 4b is an SEM image obtained at 800× magnification of a PLGA surface, which had been modified using pillars having interspacing of 150 nm, 600 nm and 1 μm, displaying a higher level of platelet adhesion on the 1 μm interspacing as compared to the 150 nm. FIG. 4c is a graph showing tabulated data from FIG. 4b obtained by visually counting platelets over an average of 6 images (n=3).

FIG. 5 is a graphical comparison between the various test substrates which are of varying aspect ratios.

FIG. 6a is an SEM image obtained at 2,000× magnification showing the lowest amount of adhered platelets (circled) on P(1,1,8) due to the high aspect ratio of the pillars. FIG. 6b is an SEM image obtained at 20,000× magnification of the region shown in FIG. 6a by the dotted box, showing the clustering of the pillars after drying.

FIG. 7a is a schematic diagram of platelet attachment on pillars with low aspect ratios. FIG. 7b is a schematic diagram of platelet inhibition on pillars with high aspect ratios.

FIG. 8a is an SEM image obtained at 2,000× magnification showing a high level of platelet adhesion on P(1,1,2) displaying an activated platelet with spike pseudopods (circled). FIG. 8b is an SEM image obtained at 15,000× magnification of the region shown in FIG. 8a by the square box, displaying a flat disc platelet.

FIG. 9a is an SEM image obtained at 2,500× magnification showing the preferential adhesion of platelets on the pristine region of PLGA substrate (left side of FIG. 9a) as compared to the region with pillars thereon (right side of FIG. 9a). FIG. 9b is an SEM image obtained at 5,000× magnification of the right side of FIG. 9a showing the clustering effect observed on the pillars after drying.

FIG. 10a is an SEM image obtained at 2,500× magnification displaying the slight elevation of adhered platelets (circled) on the 500 nm (width) pillars on P(1,5,8). FIG. 10b is a magnified SEM image obtained at 5,000× magnification of the pillars of FIG. 10a showing the reduced effect of clustered pillars after drying.

FIG. 11a is an SEM image obtained at 500× magnification of a pristine PLGA substrate showing the highest level of platelet adhesion where this morphology increase the potential for multivalent adhesive interactions by enabling maximal surface contact area with the substratum surface. FIG. 11b is an SEM image obtained at 1,000× magnification showing the platelet spreading at its advanced stage. FIG. 11c is an SEM image obtained at 2,500× magnification of the surface of a HOPG substrate showing a moderately high level of platelet adhesion. FIG. 11d is an SEM image obtained at 600× magnification of the surface of C(˜,1.5,190) displaying suppressed amount of platelet adhesion when compared to FIG. 11c, implying the influential role of surface modification in the regulation of platelet activity.

FIG. 12a is an SEM image obtained at 3,000× magnification showing the morphology of activated platelet on P(14,6,0.4). FIG. 12b is an SEM image obtained at 10,000× magnification showing the morphology of activated platelet on P(8,10,0.4).

### EXAMPLES 1 AND 2 AND COMPARATIVE EXAMPLES 1 TO 4

Non-limiting examples of the invention and comparative examples will be further described in greater detail, which should not be construed as in any way limiting the scope of the invention.

### Materials Used

### Samples

80/20 poly(lactic-co-glycolic acid) 5.01 (PLGA) was obtained from PURAC Biochem of Lincolnshire of Illinois of the United States of America. Vertically aligned carbon multi-walled nanotubes (MWNTs) were purchased from Nanolab, Inc. of Newton of Massachusetts of the United States of America. Highly Ordered Pyrolytic Graphite (HOPG) was obtained from SPI Supplies and Structure Probe of West Chester of Pennsylvania of the United States of America. Dichloromethane (DCM) was supplied from Tedia Company Inc of Fairfield of Ohio of the United States of America.

### Buffers, Biochemicals and Antibodies

Phosphate buffer saline (PBS) was made by dissolving 8.0 g NaCl, 0.2 g KH₂PO₄, 2.9 g Na₂HPO₄ and 0.2 g KCl in 1 L of de-ionized water. The washing buffer used as stop solution for enzyme linked immunosorbent assay (ELISA) consisted of 0.1% v/v Tween-20 in PBS and 0.5 M H₂SO₄. Lactate dehydrogenase (LDH) cytotoxicity assay kit was obtained from Cayman Chemical Co of Ann Arbor of Michigan of the United States of America (Cat #1008882). Lysis buffer containing 2% v/v Triton X-100 in PBS and stop solution of 1 M HCl in PBS were used for LDH assay. Calcium free tyrode buffer consisting of 4.0 g NaCl, 0.5 g NaHCO₃, 0.1 g KCl, 0.03 g Na₂HPO₄, 0.5 g D-glucose, 0.1 g MgCl₂ in 500 mL de-ionized water and 2% formaldehyde was adjusted to pH 7.4 with NaOH or HCl, as necessary.

CaCl₂, H₃PO₄, 3,3,3-trifluoropropyl-trimethoxysilane, glutaraldehyde, acetone and ethanol were all purchased from Sigma-aldrich of St. Louis of Missouri of the United States of America. Micro centrifuge tubes and 96-wells polystyrene microtiter plate were used in the incubation study and enzymatic assays respectively.

Fibrinogen from human (Hfg) was purchased from Sigma-Aldrich (˜35-65% protein, ≧95% of protein clottable). Platelet-rich plasma (PRP), horseradish peroxidase (HRP) conjugated goat antihuman fibrinogen and 3,3′,5,5′-tetramethyl-benzidene (TMB) for the ELISA tests were purchased from US Biological of Swampscott of Massachusetts of the United States of America. Fluorescently labeled IgM monoclonal antibody and procaspase activating compound-1 fluorescein isothiocyanate (PAC-1 FITC) were purchased from BD Biosciences of San Jose of California of the United States of America for flow cytometric analysis.

### Methods Used

### Fabrication of Nano Porous Anodized Alumina (NPAA) Template

To fabricate an ordered NPAA template, an indentation process was used. Here, the surface of an aluminum foil (99.999%, obtained from Alfa Asear of Ward Hill of Massachusetts of the United States of America) was indented using pressure at 4000 psi for 2 minutes with a substrate where silica colloids of particle diameter about 270 nm had been assembled. The aluminum foil was subsequently cleaned in an ultrasonic water bath to release the attachment of silica colloidal spheres on the surface. The aluminum foil was then anodized at 3° C. in 0.3 M H₃PO₄ aqueous solution, at a voltage of 112 V for a time ranging from 1 to 20 minutes depending on the desired depth of the pores. The NPAA templates had a relative constant interspacing and diameter, and the depth of the pores was controlled by varying the duration of anodization time. Typically, a depth of about 1 μm was achieved with about 20 minutes of anodization time.

### Fabrication of Silicon Master Template

The silicon master templates were fabricated using standard micro/nano fabrication techniques such as either deep ultraviolet photolithography or electron beam lithography, followed by inductively coupled plasma—deep reactive ion-etching (ICP-DRIE) BOSCH process. A variety of templates were fabricated with features ranging from nanometer to micrometer size to produce the projections with dimensions as summarized in Table 1.

### Fabrication of Projections on a PLGA Substrate

The templates as prepared above were cleaned in acetone to remove any traces of contaminants, rinsed in de-ionized water and subsequently oven dried. To facilitate the removal of casted PLGA substrate after drying, the templates were treated with an anti-adhesion agent such as 3,3,3-trifluoropropyl-trimethoxysilane for about 1 hour in the vapor-phase.

The casting mixture was prepared by dissolving PLGA pellets in dichloromethane (DCM) at a ratio of 1:28 w/v. The casting mixture was then poured onto the coated templates and vacuum baked at 70° C. for 4 days before releasing the three-dimensional features in the form of pillars or projections from the templates.

Solvent casting reproduces precise three-dimensional projections from a master template, is inexpensive and allows for reuse of the template. The pillars of PLGA were obtained from casting on several NPAA templates or silicon molds. The dimensions of the pillars fabricated were analyzed using Smile View 2 and are tabulated in Table 1, along with the corresponding aspect ratio (which is defined as the ratio of height to width).

Alternatively, capillary force imprinting can be used to form the projections on the PLGA substrates. In capillary force imprinting, PLGA, which is a thermoplastic polymer, having a thickness of 0.5 mm is placed in contact with the templates as prepared above. Subsequently, a slight contact pressure of 500 mBar was applied to the assembly to enable close contact between the polymer and mould. Following which, the temperature of the assembly was increased above the glass transition temperature of the polymer. The temperature used was about 120° C.-150° C. The viscous polymer then fills the cavities of the template driven by capillary action. The time duration of the process depends on the aspect ratio of the pores in the template and the desired size of the projections on the substrates. The replicated substrates are cooled to approximately 40° C. before removal of the template. The template can be removed by peeling off the template or by etching the template. Typical fabrication parameters are shown in Table 1.

### Fibrinogen Adsorption Determined by ELISA

Fibrinogen adsorption on the substrates was quantified by an ELISA array. A calibration curve was established first prior to quantification of the substrates. The procedure is as follows: samples of 3×3 mm substrates were first equilibrated in PBS for 30 minutes in micro centrifuge tubes. Subsequently, the samples were placed in a 96-well microtiter plate with 0.05 mg/ml of Hfg for 1 hour at 37° C. The samples were rinsed 5 times with washing buffer (0.1% v/v Tween 20 in PBS) before transferring the samples into new wells on the microtiter plate. The samples were then incubated in HRP conjugated goat antihuman fibrinogen for 1 hour at 37° C. at 1:10⁵ dilutions. The samples were rinsed subsequently for 5 times before transferring into new wells. Finally, 100 μl of a colorimetric agent such as 3,3′,5,5′-tetramethylbenzidine was added into each well and the plate was incubated in the dark for 20 minutes where the solution undergoes oxidation and turns blue upon reaction with HRP. Subsequently, 100 μl of 0.5 M H₂SO₄ was added to the wells to stop the enzymatic reaction. The solutions were quickly aspirated using a multiple pipette tool and transferred to new wells, where the absorbance values of the solutions were measured on a microtiter plate reader at 450 nm within 5 minutes. Adsorption experiments were done in triplicate (n=3) and repeated separately for 3 times.

A linear correlation (r=0.951) between the coating concentrations (0.001 to 0.05 mg/ml) of Hfg and the absorbance measured by ELISA was first obtained. The calibration curve for the absolute amount of adsorbed Hfg is expressed as:

Adsorbed Hfg (mg/mm²)=4.31×10⁻³ OD−1.489×10⁻⁴

where OD indicates the optical density or absorbance of the reaction solution as measured with the microtiter plate reader.

### Platelet Adhesion Determined by LDH Assay

A calibration curve was initially plotted by measuring the optical density (OD) values of solutions of known platelet concentration (determined by hemocytometer). The linear correlation (r=0.9882) between the measured optical density and the absolute amount of suspended adherent platelets is expressed as:

Number of adherent platelets/mm²=370.37 OD+21.26

The optical density values are primarily attributed to the suspended platelets as interaction of pre-adsorbed Hfg with the LDH reaction solution showed an insignificant absorbance value (p>0.05).

To quantify the amount of platelets adhered on the test surfaces, the substrates (of width and length of 3×3 mm) were first equilibrated with PBS for 30 minutes at 37° C. Then, 200 μl of Hfg (4 mg/ml) was added into the microtiter plate wells and incubated for 1 hr at 37° C. Subsequently, the wells were rinsed 3 times with PBS to remove any unbound Hfg. Then, 200 μl of PRP was added into the wells and the microtiter plate was incubated at 37° C. for 2 hours. After incubation, the wells were again rinsed with PBS for 3 times to remove any non-adhered platelets and the substrates were transferred into new wells. Then, 25 μl of lysis buffer was added and the microtiter plate was incubated at 37° C. for 1 hour. After that, 50 μl of LDH reaction solution was added and the microtiter plate was incubated for 20 minutes at room temperature. Afterwards, 25 μl of stop solution was added into each well and aspirated before transfer to new wells, where the OD readings were taken at 490 nm using a microtiter plate reader. Experiments were done in triplicate (n=3) and repeated separately 3 times.

### Flow Cytometry Studies

The PRP suspension extracted from the platelet-bound Hfg substrates after PRP incubation as described in the previous section was used for flow cytometry studies. The washing solution (calcium free tyrode buffer) was added to the extracted PRP and the mixture was centrifuged at 2000 rpm for 15 minutes. The supernatant was removed after centrifugation and the activated platelets were stained by direct immunofluorescence using PAC-1 FITC, in which the ratio of diluted PAC-1 FITC stain to PRP was 4:1. The stained samples were incubated at room temperature for 20 minutes in the dark. After that, an equal volume of 2% formaldehyde was added to fix the platelets. The fixed samples were stored at 4° C. in the dark and were analyzed within 18 hours.

Platelet activation, which takes place when platelets interact or attach to foreign surfaces and as a result undergo a series of complex reactions to promote thrombin formation and platelet aggregation, was quantified using a BD FACSCalibur™ flow cytometer (Becton Dickinson of San Jose of California of the United States of America) equipped with 488 nm laser excitation wavelength. A total of 10,000 platelets were gated per sample from the dot plot which was set in the forward side scatter (FSC) and side scatter light (SSC) channels to obtain the mean intensities of fluorescence for platelet-bound Hfg. The fluorescence intensity was collected through a 530/30 (FL1) bandpass filter. Logarithmic amplification was used due to the small platelet size. The data obtained from flow cytometry were re-analyzed with WinMDI 2.9 software package. (http://en.bio-soft.net/other/WinMDI.html)

### Observation of Adhered Platelet on Substrates by FESEM

The substrates prepared according to the section titled “Platelet Adhesion Determined by LDH Assay” were fixed with 2.5% glutaraldehyde for 2 hours at 4° C. Consequently, the substrates were rinsed in PBS and dehydrated through a series of graded alcohols (0%, 25%, 50%, 75%, 100%) before they were freeze dried at −80° C. and platinum coated for observation under the field emission scanning electron microscope (FESEM) (JEOL JSM-6340F) for the changes in the platelet morphology.

### Statistical Analysis

All experiments were run in triplicate and repeated separately for at least three times. The PRP and plasma are stored at −20° C. when not in use and are stable up to 12 months. Data obtained from the different assays are presented as mean values with standard deviations. Statistical significance of differences between two substrates was analyzed using single factor analysis of variance (ANOVA).

### Results

### Characteristics of PLGA Substrates

The PLGA substrates prepared using the method discussed above present three-dimensional projections in the form of pillars that extend from the surface. The dimensions and inter-pillar spacing of these pillars for examples 1 and 2 as well as comparative examples 1 to 4 are presented in Table 1 below.

TABLE 1

| | | Interspacing | Width | Height | Aspect | Adhesion |
|---|---|---|---|---|---|---|
| | Substrate | (I) nm | (W) nm | (H) nm | Ratio | Level |
| Example 1 | P (1, 1, 2) | 120 ± 30 | 100 ± 30 | 200 ± 70 | ~2 | High |
| | | | | | | adhesion |
| Comparative | P (1, 1, 8) | 130 ± 30 | 100 ± 30 | 800 ± 130 | ~8 | Low |
| Example 1 | | | | | | adhesion |
| Example 2 | P (1, 5, 8) | 140 ± 30 | 500 ± 10 | 840 ± 160 | ~1.7 | High |
| | | | | | | adhesion |
| Comparative | P (1, 2.5, 8) | 140 ± 30 | 250 ± 10 | 840 ± 160 | ~3.4 | Low |
| Example 2 | | | | | | adhesion |
| Comparative | P (8, 10, 0.4) | 840 ± 10 | 1000 ± 60 | 40 ± 10 | ~0.04 | High |
| Example 3 | | | | | | adhesion |
| Comparative | P (14, 6, 0.4) | 1440 ± 20 | 600 ± 60 | 40 ± 10 | ~0.07 | High |
| Example 4 | | | | | | adhesion |
| Reference | C (~, 1.5 ,190) | ≠ | 150 ± 50 | 19000 ± 5800 | ~130 | — |
| Example | | | | | | |

Substrate notation: for example, (1, 1, 2) refers to (interspacing, width height) of the pillars expressed in 10² nm.
≠ Not measurable due to high density of carbon nanotubes

### Platelet Adhesion and Activation on PLGA Substrates

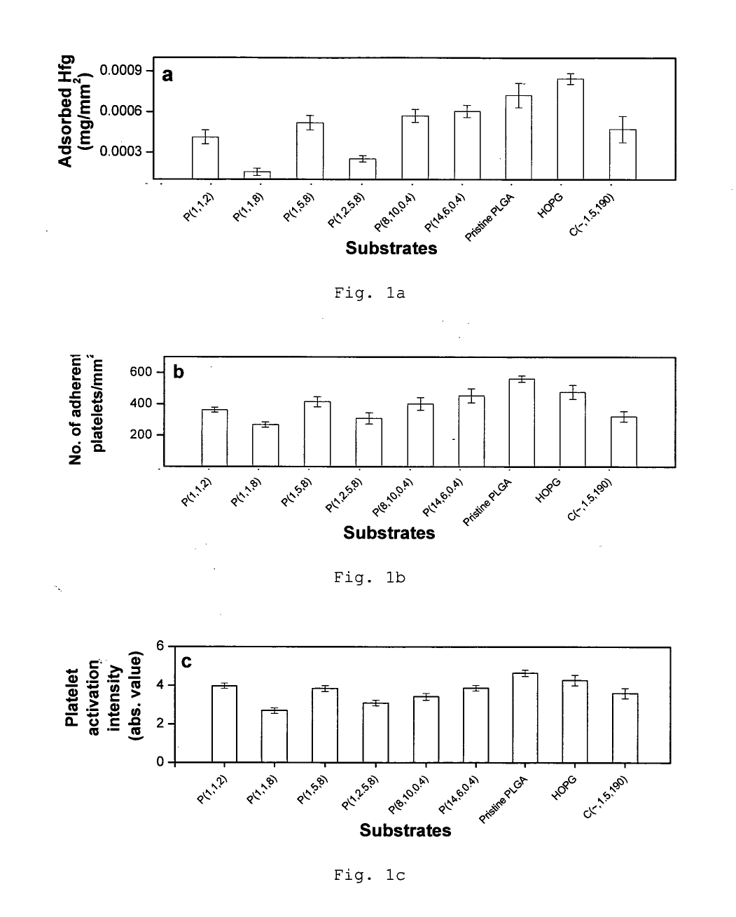

In order to determine the level of platelet adhesion on the PLGA substrates with topographic projections, a reference sample such as a silicon substrate having vertically aligned multi wall carbon nanotubes (MWNTs) on the surface was used to examine the effect of topographic projections. Highly ordered pyrolytic graphite (HOPG) sheets were also used as another sample for comparison purposes. The parameters of this reference sample are depicted in Table 1 above. This reference sample is used to determine the extent of adhesion on the test polymer substrates. As shown in FIG. 1b, any polymer substrates having a lower level of platelet adhesion as compared to the reference sample will be represented in Table 1 as having a “low adhesion”. As such, any polymer substrates having a higher level of platelet adhesion as compared to the reference sample will be represented in Table 1 as having a “high adhesion”.

FIG. 1a and FIG. 1c show the level of adsorbed Hfg and the extent of platelet activation, respectively, on the PLGA substrates, pristine PLGA substrate, HOPG substrate and the reference sample. It can be seen that the level of adsorbed Hfg and extent of platelet activation for Examples 1 and 2 are lower as compared to those of the reference sample.

The statistical significant correlations between substrates performed by ANOVA are tabulated from FIG. 1a to FIG. 1c and are shown in Table 2 below. Table 2 displays (a) the amount of adsorbed fibrinogen, (b) the number of adherent platelets and (c) platelet activation intensity. Data were run in triplicate (n=3) and expressed as mean±standard deviations of 3 repeated independent experiments.

TABLE 2

| | | | | | | | | | | | | | | | | Pristine | | | | | | | | |
|---|---|---|---|---|---|---|---|---|---|---|---|---|---|---|---|---|---|---|---|---|---|---|---|---|
| | P(1, 1, 8) | | | P(1, 5, 8) | | | P(1, 2.5, 8) | | | P(8, 10, 0.4) | | | P(14, 6, 0.4) | | | PLGA | | | HOPG | | | C(~, 1.5, 190) | | |
| Substrate | a | b | c | a | b | c | a | b | c | a | b | c | a | b | c | a | b | c | a | b | c | a | b | c |
| P(1, 1, 2) | ¤ | ¤ | * | * | * | ≠ | ¤ | * | ¤ | ¤ | * | * | ¤ | ¤ | ≠ | ¤ | ¤ | ¤ | ¤ | ¤ | ≠ | ≠ | * | ≠ |
| P(1, 1, 8) | | | | ¤ | ¤ | * | ¤ | * | * | ¤ | ¤ | ≠ | ¤ | ¤ | ≠ | ¤ | ¤ | * | ¤ | ¤ | * | ¤ | * | ≠ |
| P(1, 5, 8) | | | | | | | ¤ | ¤ | ¤ | ≠ | ≠ | * | * | ≠ | ≠ | ¤ | ¤ | ¤ | ¤ | * | * | ≠ | ¤ | ≠ |
| P(1, 2.5, 8) | | | | | | | | | | ¤ | * | ¤ | ¤ | ¤ | ¤ | ¤ | ¤ | ¤ | ¤ | ¤ | ¤ | ¤ | ≠ | ¤ |
| P(8, 10, 0.4) | | | | | | | | | | | | | ≠ | ≠ | * | * | ¤ | ¤ | ¤ | * | * | * | * | ≠ |
| P(14, 6, 0.4) | | | | | | | | | | | | | | | | * | ¤ | ¤ | ¤ | ≠ | ≠ | * | ¤ | ≠ |
| Pristine PLGA | | | | | | | | | | | | | | | | | | | * | * | ≠ | ¤ | ¤ | * |
| HOPG | | | | | | | | | | | | | | | | | | | | | | * | ¤ | * |

≠ denotes non-significance,
* denotes significance for (p < 0.05),
¤ denotes significance for (p < 0.001).

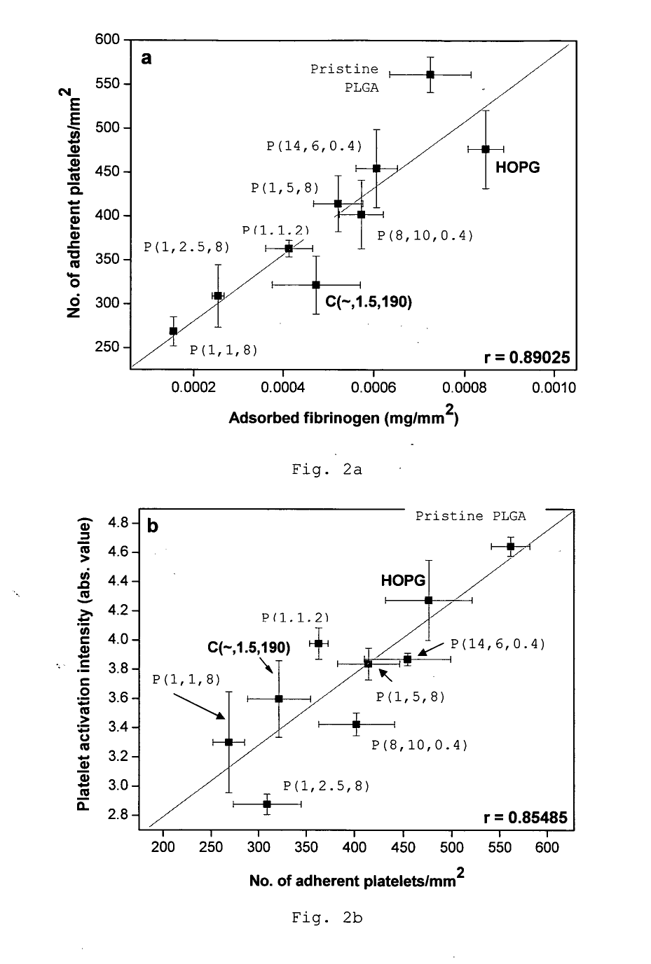

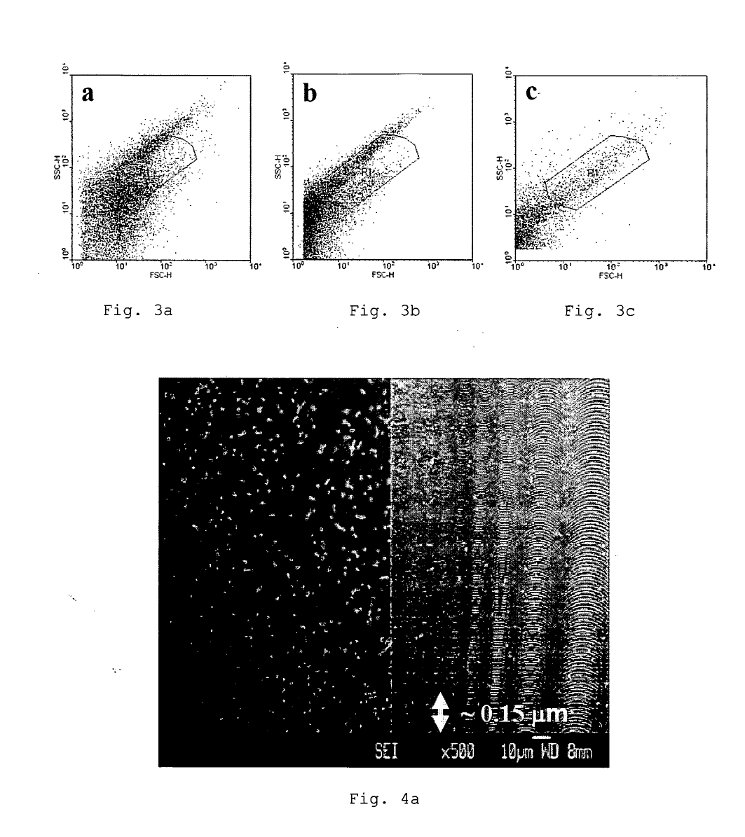

As shown in FIG. 1a and FIG. 1b, different amount of Hfg adsorption was observed on the PLGA substrates with projections of varying dimensions and interspacing, and the result is relatively well-correlated to the subsequent amount of platelet adhesion on the substrates. A correlation plot on the amount of platelet adhesion to the adsorbed Hfg shows a strong correlation (r=0.88876) as shown in FIG. 2a. This means that the data for both tests done on the same topographic projections matches well which, proves the effectiveness of the specific projections. The resultant r²=0.789 indicates that the adherence of the platelets is mostly dependent on fibrinogen adsorption (about 79%) but that other factors (about 21%) such as variation in surface chemistry (e.g., HOPG vs. PLGA) also play a part.

However, if data based on PLGA samples only is used, a stronger correlation is seen (r²=0.93) (data not shown), indicating that once surface chemistry is fixed; fibrinogen adsorption differences dictate the subsequent platelet adhesion amount on those structured PLGA surfaces.

A strong correlation was also observed for the number of adherent platelets determined by the LDH assay against the PAC-1 expression for activated platelets determined by flow cytometric analysis (r=0.84492) in FIG. 2b. This means that the data for both tests done on the same topographic projections matches well which, proves the effectiveness of the specific projections. The related r² value of 0.714 indicates that about 71% of the variance in platelet activation can be attributed to the amount of adherent platelets and that about 29% of variability in the platelet activation quantity is unexplainable at this point. Similarly, a correlation study of the same correlation between the number of adherent platelets against the PAC-1 expression for activated platelets on all the PLGA substrates (i.e. textured and pristine PLGA) displayed a slightly stronger correlation (r²=0.72) (data not shown).

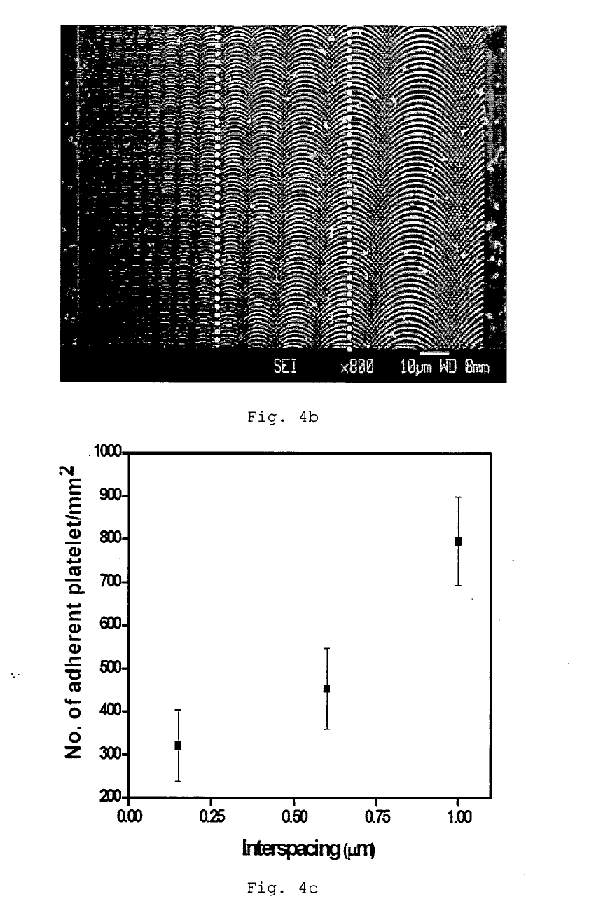

As shown in FIG. 3, the activated platelet population was gated and defined by flow cytometric region (R1) according to their size and morphological characteristics. A higher platelet activation was observed on pristine PLGA surface as compared to that of P(1,2.5,8) with a lower mean intensity.

As demonstrated above, the degree of platelet response and Hfg adsorption amount were clearly different on the structured PLGA films whereby markedly reduced levels of adsorbed Hfg and platelet response were observed on all the structured PLGA films when compared to that of pristine surfaces. Correspondingly, statistically significant differences were observed in Hfg adsorption and platelet response when comparing the structured PLGA substrates to that of pristine surfaces in terms of projection size, aspect ratio and density in all three experimental studies, indicating the importance of these parameters in influencing the hemocompatibility of a surface.

### Interspacing Effect

To study the effect of interspacing on platelet adhesion, two PLGA substrates were studied here. As shown in FIG. 4a and FIG. 4b, these figures clearly reveal a reduced level of platelet adhesion on the region with about 150 nm interspacing and a high level of adherent platelets on the region with about 1000 nm interspacing. In FIG. 4b, the substrate is split into three regions in which the vertical interspacing of the structures in the region on the left-hand side of the substrate is 150±10 nm; the vertical interspacing of the structures in the region in the middle of the substrate is 580±40 nm; and the vertical interspacing of the structures in the region on the right-hand side of the substrate is 9500±50 nm. The horizontal interspacing of the structures is maintained at 125±40 nm for all three regions. The width of the structures is maintained at 500±10 nm for all three regions. The height of the structures is maintained at 450±150 nm for all three regions.

As shown in FIG. 4c, when the interspacing becomes less than about 200 nm, adhesion is clearly reduced due to the decreased area of contact between the projections and the platelets. As such, the conditions are not favorable for the platelets to establish stable contact with the PLGA surface and to acquire the right cellular morphological phenotype, which leads to activation. Low level of platelet activation was therefore observed when the inter-pillar spacing is reduced as the platelet morphology becomes restricted by its inability to contact the entire surface area. Hence, based on the results from FIG. 4c, an interspacing of less than or about 200 nm appears to be effective in minimizing platelet adhesion and hence, platelet activation, on these structured surfaces.

In order to reduce the probability of platelet entrapment between the projections and the formation of stable contact with the surface which leads to platelet activation, the dimension of the interspacing should be much smaller than the platelet size.

### Aspect Ratio Effect

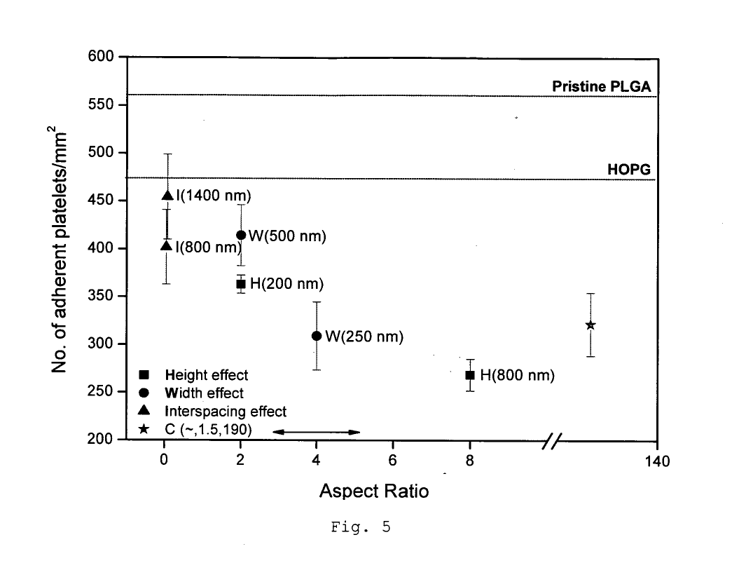

FIG. 5 shows the tabulated results from FIG. 1b of platelet adhesion per area versus the aspect ratio of the PLGA substrates tested. In FIG. 5, the PLGA substrates represented by the label “height effect” are Example 1 and Comparative Example 1; the PLGA substrates represented by the label “width effect” are Example 2 and Comparative Example 2; and the PLGA substrates represented by the label “interspacing effect” are Comparative Examples 3 and 4. It can be seen that platelet adhesion was markedly decreased on substrates with increasing aspect ratio. An aspect ratio of 3 to 5 (represented by the arrow in FIG. 5) appears appropriate to realize a sufficiently low amount of adherent platelets. However, the aspect ratio of the projections can be greater than this range as long as the projections do not cluster too much such that they intertwine each other to form an intertwined structure that offers a greater contact surface area for the platelets to adhere to. The dotted lines depicted in FIG. 5 refer to controls used in this study.

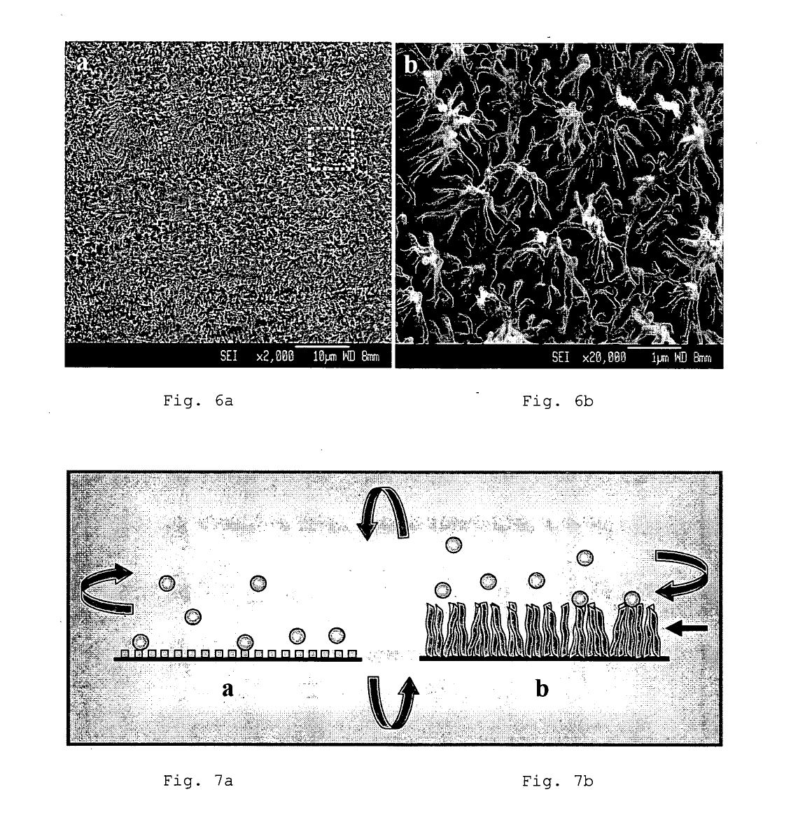

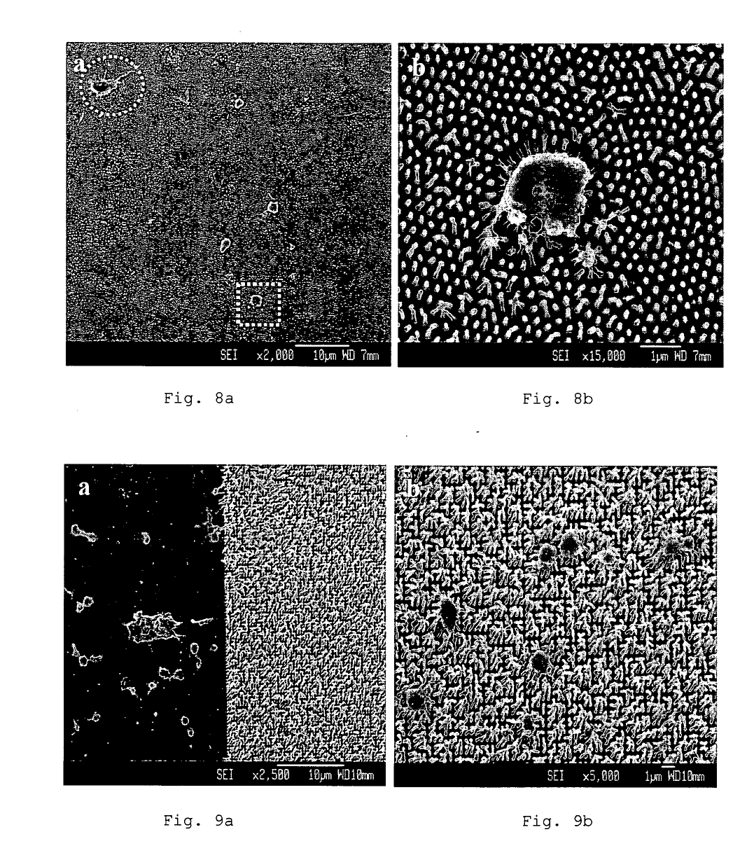

It appears that platelets are able to interact only with the tips of the high aspect ratio projections when interspacing becomes sufficiently small (i.e. about less than or equal to 200 nm). As shown in FIG. 6a, projections with high aspect ratio such as in the substrate P(1,1,8) are likely to get deformed and bent in a liquid media even under slight agitation during incubation. This flexibility of the projections results in any unstable adhesion of adherent platelets and the inability to acquire the right cellular morphological phenotype, hence the platelets can be dislodged easily from the projection's surface. This effect is illustrated in FIG. 7a and FIG. 7b. FIG. 7a is a schematic diagram illustrating that projections having a low aspect ratio will remain stiff and upright in a suspension, thereby permitting more platelet attachment at a greater ease and frequency as compared to the scenarios in FIG. 7b. FIG. 7b is a schematic diagram showing the projections with high aspect ratios in two proposed scenarios (i) flexible pillars due to their bending ability act to prevent the platelets in making a successful contact on the projections and (ii) the possible detachment of any unstable adherent platelets. The arrows present in FIG. 7a and FIG. 7b indicate the frictional forces is from the fluid in motion.

As shown in FIG. 6b and FIG. 8b, the differences in platelet adherence as observed on P(1,1,8) in FIG. 6b when compared to P(1,1,2) in FIG. 8b are obvious with tabulated data showing high significance (p<0.001). The clustering of the pillars observed in FIG. 6b was due to capillary forces while drying the sample for electron microscope observation it was presumed that these pillars would exist as individually separated entities in liquid phase during the PRP incubation. The inventors surmised that the impaired platelet response on P(1,1,8) could be also due to the low level of adsorbed Hfg measured or inadequate conformational change adopted on these surfaces. Both effects, i.e. surface modification and fibrinogen structural changes, may act jointly to decrease the adhesion of platelets. Hence, an aspect ratio value of in the range of 2.5 to 5 appears to be sufficient to reduce a significant amount of adherent platelets on the surfaces.

### Width Effect

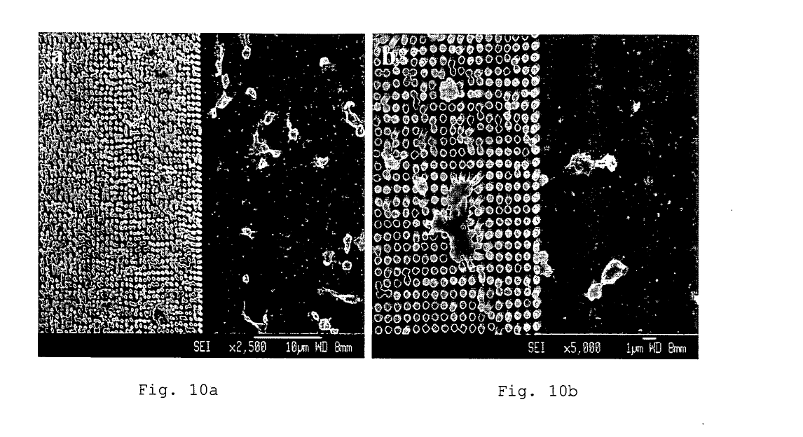

A range of projections having widths from 100 nm to 3000 nm were studied for platelet response in this study. The amount of adsorbed Hfg and adherent platelets with its subsequent activation were observed to be significantly lower (p<0.001) on the substrate (P(1,2.5,8)) with projections having a width of 250 nm (FIG. 9a, 9b) as compared to the substrate having projections with a width of 500 nm (P(1,5,8)) (FIG. 10a, 10b). In comparing P(1,2.5,8) with P(1,5,8), the fibrinogen adsorption differences are statistically significant, and hence, these differences are responsible for the observed increased adsorption on the P(1,5,8) surface. In other words, on the wider pillars, fibrinogen adsorption is increased, perhaps due to the greater area of contact. Subsequently, platelet adherence is also increased. Likewise, the same statistical significance was also observed on 100 nm width pillars in P(1,1,8) when compared to that of 500 nm wide pillars in P(1,5,8), with reduced Hfg and platelet response observed on P(1,1,8).

Additionally, when comparing between substrates P(1,1,2) and P(1,5,8), even though these substrates have approximately similar aspect ratio of about 2 in FIG. 5, it can be seen that the level of adherent platelets was noticeably lesser on P(1,1,2). These results indicate that the absolute dimensions of the pillars are also important in the reduction for platelet activity in addition to the aspect ratio. It can be envisaged that pillars with an enlarged width act to increase the surface contact area for enhanced fibrinogen adherence and consequently, enhanced platelet adherence/interactions by allowing adhesion to occur with greater ease.

Chemical versus Effect Due to Projections on Surface

The highest level of Hfg adsorption and platelet response was observed markedly on the pristine surfaces without any projections thereon, e.g. PLGA and HOPG. This is clearly due to the fact that fibrinogen or platelet adsorption on such surfaces is conformationally optimum, and not constrained by the presence of physical features. In order to further investigate the impact of projections on platelet response, the planar carbon surface of HOPG was evaluated versus its structured equivalent, on the MWNTs, C(˜,1.5,190). The surface chemistry of these substrates is similar.

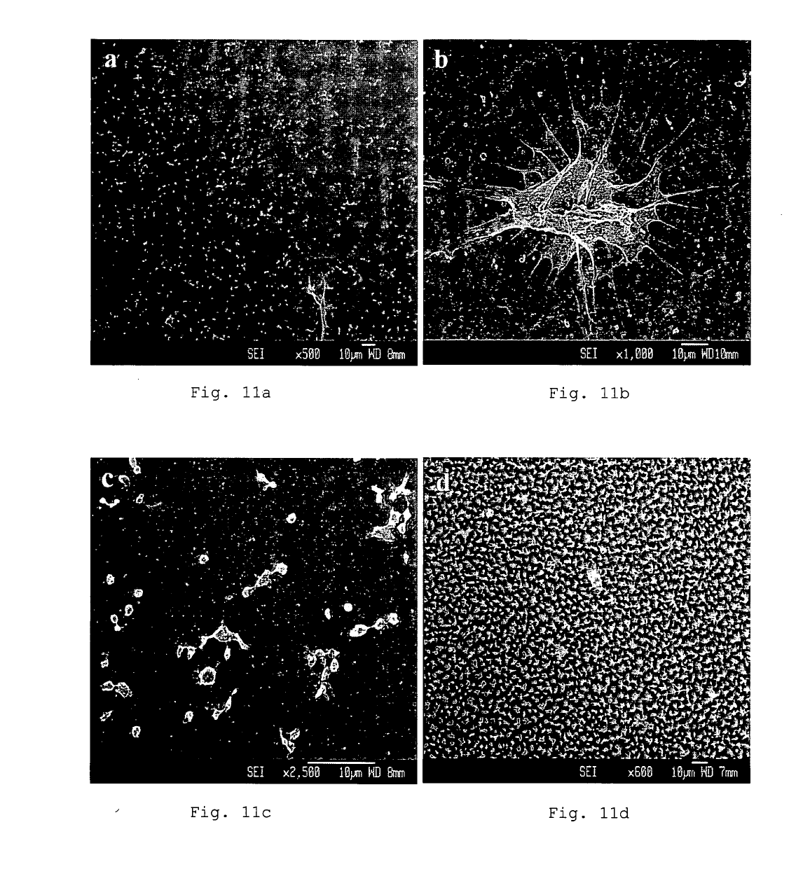

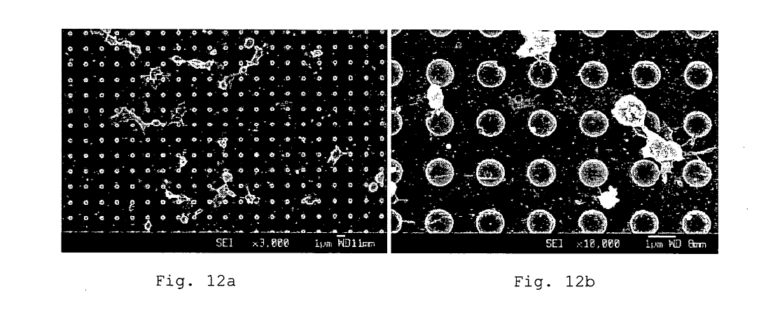

Here, the results showed statistically significant differences between HOPG and C(˜,1.5,190) in all three aspects (i.e. adsorbed Hfg, adherent and activated platelet), inferring the influential role of surface modification. However, the chemistry of the surface is also important, as seen in the differences in the behavior of C(˜,1.5,190) and the structurally-modified PLGA films. The chemistry of the surface needs to be taken into account since the initial adhesion of proteins would be quite different due to the protein-surface binding affinity. The platelet adhesion and activation on the pristine PLGA (FIGS. 11a and 11b) is clearly the highest in all the tests performed on pristine PLGA, followed by pristine HOPG (FIG. 11c). This exemplifies the effect of surface chemistry on adhesion between PLGA and HOPG, where statistically significant difference (p<0.05) was observed in the Hfg adsorption and adherent platelets. However, it is noted that there was no statistical differences in the platelet activation as seen in Table 2. This result indicates that the chemistry of surfaces influences differentially during the initial adsorption and binding stages but not in the later stage of platelet activation.

Compared to the pristine surfaces, platelet response was evidently lower on the structured PLGA and carbon substrate, in this case C(˜,1.5,190). However, the adhesion level on C(˜,1.5,190) (FIG. 11d) is comparable to that of high aspect ratio structured PLGA films (FIG. 1b). Hence this indicates that the chemical influence on adhesion can be overcome by suitable structural modifications of a surface. However, it may be possible that both effects of structural modification and chemistry may act together to determine the platelet response on artificial surfaces.

Hence, in addition to surface chemistry, surfaces with specific structural features may result in reduced thrombogenicity.

### Morphology of Activated Platelets

The morphologies of activated platelets are classified into 5 categories from the lowest to the highest level of activation including: round, dendritic, spread-dendritic, spread and fully spread.

The least amount of platelet activation observed on the substrate with a high aspect ratio P(1,1,8) has a round platelet morphology (FIG. 6a), followed by P(1,2.5,8) with reduced dendritic morphology as shown in FIG. 9b compared to that of a larger pillar width, P(1,5,8) with a spread-dendritic morphology (FIG. 10b). The result shows that platelet activation is increased as the pillar's width size becomes larger since the potential for multivalent adhesive interactions on the platelet is increased with an enlarged surface contact area.

Conversely, a substrate with a low aspect ratio, P(1,1,2), displayed high level of activation as platelets lose their discoid shape and became extended with spiky pseudopods adopting a spread morphology (FIG. 12a and FIG. 12b).

The activation level on the pristine PLGA is clearly the greatest (FIG. 11a) where the platelets are fully spread as the cytoplasm expands through the surface causing the platelets to spread into a thin film with the continual adherence of platelets (FIG. 11b). In comparison, the platelet activation level on HOPG (FIG. 11c) is relatively lower and adopts the reduced spread morphology highlighting the low thrombogenicity of this material.

The activation level on the MWCN (C(˜,1.5,190)) in FIG. 11d and P(1,5,8) in FIG. 6a are comparable, both showing reduced spread-dendritic morphology compared to that of a bigger interspacing substrate, P(14,6,0.4)in (FIG. 12a and FIG. 12b). The result reveals that platelet activation increases with increased interspacing between the structural features, attributed particularly to the increased ability of the platelet to interact with the surface as the effective contact surface area increases.

The results presented underline the importance of an appropriate dimensional control of structured surfaces in directing a desired platelet response. Structural features with reduced interspacing (such as shown in FIG. 4b), reduced width (such as in P(1,2.5,8)) and increased aspect ratio (such as in P(1,1,8)) were found to result in a significant reduction of adsorbed Hfg and platelet response. In reducing the risk of clot formation, not only is it important to reduce the amount of fibrinogen adsorbed to a surface, but also to influence the adopted conformation on the surface as it determines the subsequent platelet adhesion/activation. The geometrical features of the projections play an important role in this. In addition, carbon based surfaces in the form of MWNTs become minimally thrombogenic when compared to a thromboresistant material such as HOPG.

### CONCLUSIONS

Platelet adhesion and activation have been shown by the above examples and comparative examples to be influenced by projections that extend from the substrate surface at the nanometer level with the significant reduction of platelet response compared to that of controls.

In conclusion, it was found that:

(a) On pristine surfaces, surface chemistry plays a dominant role on adhesion, as shown by the reduced fibrinogen and platelet adherence on HOPG as compared to PLGA.

(b) When surfaces are structured, these features are able to overcome the chemical influence. For example, a submicron structured PLGA surface can exhibit lowered platelet adherence than the pristine PLGA and HOPG.

(c) Tailoring surfaces with suitable structural features in addition to a specific surface chemistry acts to reduce the platelet response.

(d) The specific dimensions of the projections have a significant effect. Interspacing, width and height (i.e. aspect ratio) have been observed to significantly influence the platelet adherence and activation, in the following manner:
Increased aspect ratio in submicron projections reduces the number of adherent platelets due to the inability of the platelets to form stable contacts with the reduced surface area for attachment.
Increasing the width increases the likelihood of platelet adherence, due to the increase in surface contact area and presumably via the conformation of the adsorbed Hfg.
Increased interspacing of dimension greater than 200 nm results in the entrapment of platelets and its subsequent activation due to increased surface area of contact and entrapment.

In conclusion, the presence of structural features on a surface can add another level of control to reduce the platelet-biomaterial interactions and will be beneficial in the development of low-thrombogenic surfaces for blood contacting medical devices. By merely physically altering the surface, the degree of platelet adhesion can be reduced to much lower, levels to that observed on the “minimally thrombogenic” surfaces such as HOPG.

### APPLICATIONS

The disclosed substrate can be used in a variety of medical devices whereby the device is required to be in contact with a bodily fluid. For example, these devices can include medical or veterinary implants such as pacemakers, ear implants, nose implants, grafts or protheses; devices in contact with blood such as heart valves, cardiovascular graphs, shunts or stents; as well as devices handling or in contact with other bodily fluids and tissues such as catheters or drainage tubes.

The disclosed substrate can be used to substantially inhibit adhesion of a target cell such as microorganisms such as bacteria or fungi as well as biological cells such as blood cells, platelets or leucocytes.

The disclosed substrate may not require a chemical treatment or be coated with a chemical substance in order to modify the surface of the substrate. As such, the biocompatibility and surface chemistry of the disclosed substrate may not be altered significantly. Further, as the projections are formed on the disclosed substrate as an integral unit, the projections may not be dislodged easily from the substrate and may be resistant to the shear forces that may be present when the substrate is placed in a biological system (for example, in the blood stream of a patient).

The projections extending from a surface of the disclosed substrate may have dimensions and inter-projections spacing that are regular and consistent between batch-to-batch substrates as a result of the method used to form the disclosed substrates. As such, the disclosed substrates are reproducible and the extent of platelet adhesion can be predicted with a certain level of certainty. Further, the disclosed method may be capable of forming projections that are in the nano-scale range in order to decrease the level of adhesion of a target cell thereon.

The disclosed substrate may be capable of substantially decreasing the level of adhesion of a target cell, as compared to conventional substrates, as a result of the projections having heights, widths, aspect ratios and inter-projection spacing as mentioned above.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A substrate having a surface for inhibiting adhesion of a target cell thereon, the substrate comprising an array of generally longitudinal projections having a longitudinal axis that extends from the surface of said substrate and having an aspect ratio of at least 2.5, wherein adjacent projections of said array are configured on the substrate such that the projections at least partially inhibit adhesion of a target cell thereon.

2. The substrate as claimed in claim 1, wherein adjacent projections of said array are disposed from each other at a distance less than the diameter of the target cell.

3. The substrate as claimed in claim 1, wherein adjacent projections of said array are disposed from each other at a distance of 200 nm or less.

4. The substrate as claimed in any one of the preceding claims, wherein the aspect ratio of said projections is at least 3.

5. The substrate as claimed in any one of the preceding claims, wherein the aspect ratio of said projections is in the range of 3 to 20.

6. The substrate as claimed in any one of the preceding claims, wherein at least one of the substrate and projections are comprised of a biocompatible polymer.

7. The substrate as claimed in claim 6, wherein the biocompatible polymer is thermoplastic.

8. The substrate as claimed in claim 7, wherein the thermoplastic is selected from the group consisting of poly(D,L-lactic acid) (PDLLA), polyglycolic acid (PGA), poly(3-hydroxybutyrate) (PHB), polycarbonate (PC), poly(lactide-co-glycolic acid) (PLGA) Polycaprolactone (PCL), Poly(ethylene terephthalate) (PET), Polypropylene (PP), Polyurethanes (PU), Poly(vinyl chloride) (PVC) Polysulfones (PS) Polyamides (PA) Polyacetal (POM), Polyacrylonitrile (PAN), Poly(tetrafluoroethylene) (PTFE), polydimethylsiloxane (PDMS), epoxies and combinations thereof.

9. The substrate as claimed in any one of the preceding claims, wherein said target cell is selected from the group consisting of thrombocytes, macrophages, dendritic cells, lymphocytes, granulocytes and bacteria.

10. The substrate as claimed in any one of the preceding claims, wherein the projections are flexible.

11. The substrate as claimed in any one of the preceding claims, wherein the projections have heights in the range of from 300 nm to 8000 nm.

12. The substrate as claimed in claim 11, wherein the projections have heights in the range of from 300 nm to 800 nm.

13. The substrate as claimed in any one of the preceding claims, wherein the projections have widths in the range of from 80 nm to 800 nm.

14. The substrate as claimed in claim 13, wherein the projections have widths in the range of from 100 nm to 200 nm.

15. The substrate as claimed in any one of the preceding claims, wherein the longitudinal axis of the projections are disposed at a substantially normal angle relative to a planar surface of said substrate.

16. A implantation device for implantation into a patient's body, wherein the device comprises a surface that has an array of longitudinal projections extending from a surface of said device and wherein said projections have an aspect ratio of at least 2.5, wherein adjacent projections of said array are configured on the surface of the device such that the projections at least partially inhibit adhesion of a target cell thereon.

17. A method of preparing a surface of a substrate, the method comprising the step of forming an array of longitudinal projections that extend from the surface of said substrate and have an aspect ratio of at least 2.5, wherein adjacent projections of said array are at a configuration on the substrate to at least partially inhibit adhesion of a target cell thereon.

18. The method as claimed in claim 17, wherein the step of forming an array of longitudinal projections comprises the step of nanoimprinting the projections on the substrate.

19. The method as claimed in claim 17, wherein the step of forming an array of longitudinal projections comprises the steps of:
a) providing a mold having longitudinal cavities extending therethrough;
b) applying a thermoplastic material to the mold and allowing the thermoplastic material to enter said longitudinal cavities under capillary force to thereby produce said projections on the substrate.

20. The method as claimed in claim 19, wherein the temperature of the thermoplastic material during said applying step is above the glass transition temperature of the thermoplastic material.

21. The method as claimed in claim 17, wherein the step of forming an array of longitudinal projections comprises the steps of:
a) providing a mold having longitudinal cavities extending therethrough;
b) applying a polymerizable solution to the mold and allowing said polymerizable solution to enter said longitudinal cavities;
c) heating the polymerizable solution after said applying step (b), to thereby produce said projections on the substrate.

22. The method as claimed in claim 17, wherein the step of forming an array of longitudinal projections comprises the steps of:
a) providing a mold having longitudinal cavities extending therethrough;
b) applying a polymer solution to the mold and allowing said polymerizable solution to enter said longitudinal cavities;
c) evaporating the solvent solution after said applying step (b), to thereby produce said projections on the substrate.

23. The method as claimed in claim 17, wherein at least one of the substrate and projections are comprised of a biocompatible polymer.

24. A method of inhibiting adhesion of a target cell on a device in contact with body fluids, the method comprising the steps of:
providing a device comprising a substrate having an array of longitudinal projections extending from the surface of said substrate and having an aspect ratio of at least 2.5, wherein adjacent projections of said array are configured on the substrate such that the projections at least partially inhibit adhesion of a target cell thereon; and
contacting the body fluids with the substrate.
